(19) United States
(12) Patent Application Publication
(10) Pub. No.: US 2011/0009645 A1
(43) Pub. Date: Jan. 13, 2011

(54) MEMBRANE SEPARATION METHOD FOR SEPARATING HIGH BOILER DURING THE PRODUCTION OF 1,3-DIOXOLANE-2-ONES

(75) Inventors: Stefan Birnbach, Dirmstein (DE); Hans Klink, Hessheim (DE); Hans-Martin Mugrauer, Mutterstadt (DE); Hartwig Voss, Frankenthal (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/867,503

(22) PCT Filed: Feb. 27, 2009
(86) PCT No.: PCT/EP09/52354

§ 371 Date: Aug. 13, 2010

(30) Foreign Application Priority Data
Feb. 29, 2008 (EP) 08152146.0

(51) Int. Cl. C07D 317/38; C07D 317/36

(52) U.S. Cl. 549/230

(57) ABSTRACT

The present invention relates to a process for the continuous preparation of a 1,3-dioxolan-2-one wherein a discharge from the reaction zone is subjected to a fractionation by means of a semipermeable membrane in order to separate off polymeric by-products.

The present invention relates to a process for the continuous preparation of a 1,3-dioxolan-2-one wherein a discharge from the reaction zone is subjected to a fractionation by means of a semipermeable membrane in order to separate off polymeric by-products.

The preparation of 1,3-dioxolan-2-ones such as ethylene carbonate or propylene carbonate by reacting a corresponding oxirane (e.g. ethylene oxide or propylene oxide) with carbon dioxide in the liquid phase in the presence of a catalyst dissolved homogeneously in the liquid phase is known. Such a process is, for example, described in DE 19819586 A1. The work-up of the reaction discharge, i.e. the isolation of the product and the removal of the catalyst for recirculation to the reaction zone, is carried out by known methods such as distillation, extraction or stripping. A customary procedure comprises separating off low boilers and product by distillation and subsequently recirculating the catalyst-comprising bottom products to the reaction. A disadvantage of this procedure is that high-boiling by-products of the reaction, e.g. the cyclic and linear polyethers resulting from the oxiranes used, accumulate in the reaction system. These high boilers, which can have molecular weights up to about 20 000 dalton, lead to an increase in viscosity in the catalyst recycle stream. In, for example, the work-up by distillation, the distillation bottoms enriched in high boilers therefore has to be removed from the system together with the catalyst present therein at regular intervals. This leads to adverse effects on the economics of the process due to the required downtime of the plant and also the loss of catalyst and product occurring with the disposal of high boilers. There is therefore a need for a process for the continuous preparation of 1,3-dioxolan-2-ones, which does not have these disadvantages.

The use of semipermeable membranes for separating off homogeneous catalysts in continuous syntheses is known. Such processes are described, for example, in DE 10328713 A1 and DE 10328715 A1. However, these processes are intended to solve a chemically different problem, namely the addition of two terminal olefins which bear at least two functional groups. In the work-up of the reaction discharge from the preparation of 1,3-dioxolan-2-ones, the high boilers are obtained in admixture with the 1,3-dioxolan-2-ones which are generally also used as reaction solvent and the catalyst. Since the formation of 1,3-dioxolan-2-ones is an equilibrium reaction unlike the chemistry described in DE 10328713 A1 and DE 10328715 A1, irreversible blocking of the membrane caused by the oxirane formed in the backreaction (e.g. by reaction of the oxirane with reactive functional groups on the surface and/or in the pores of the membrane) or by deposition of polymeric products of the oxirane on and in the membrane has to be expected in this case. Furthermore, it would have been expected that chain extension of the polymers which have deposited on the surface and/or in the pores of the membrane by further addition of oxirane would play a negative role in respect of irreversible blocking of the membrane.

It has now surprisingly been found that it is possible to separate off the high-boiling (polymeric) by-products from the reaction discharge of the 1,3-dioxolan-2-one preparation by means of a membrane separation process. It is particularly surprising that separation of the high boilers from the retentate of the membrane separation is possible without an appreciable reduction in the permeability of the membrane occurring as a result of deposition or buildup of polymers on the membrane surface and/or in the membrane pores. The process of the invention is thus also advantageous for the continuous removal of the polymeric by-products formed. Both regular downtime of the plant and an uneconomical loss of catalyst can be avoided.

The invention accordingly provides a process for the continuous preparation of a 1,3-dioxolan-2-one of the general formula I

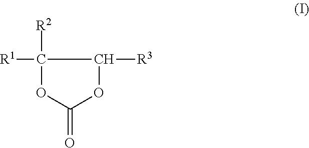

where
R¹ is hydrogen or an organic radical having from 1 to 40 carbon atoms,
R² and R³ are each, independently of one another, hydrogen or C₁-C₄-alkyl, where R² and R³ may also be joined to one another to form a five- or six-membered ring, wherein:
a) an oxirane of the general formula II

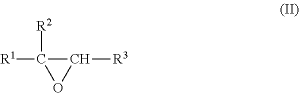

where R¹, R² and R³ are as defined above, is reacted with carbon dioxide in the liquid phase in the presence of a catalyst dissolved homogeneously in the liquid phase in a reaction zone,
b) a liquid discharge comprising polymeric by-products of the reaction is taken from the reaction zone and subjected to a work-up comprising fractionation by means of a semipermeable membrane to give a permeate and a retentate, with a high molecular weight fraction of the polymeric by-products being retained by the membrane, and
c) a purge stream comprising the high molecular weight fraction of the polymeric by-products is provided from the retentate.

The preparation of the compounds (I) leads to formation of high-boiling by-products which are essentially oligomers and polymers derived from the oxiranes (II) used for the reaction. These oligomers and polymers can be both linear and cyclic compounds. These by-products are summarized for the purposes of the present invention by the term “polymeric by-products”.

Due to the continuous manner in which the reaction is carried out, an increase in the molecular weight of the polymeric by-products comprised in the discharge from the reaction zone initially occurs at the beginning of the process of the invention until the separation limit of the membrane(s) used is reached. After this, an essentially steady state is established due to the removal of the high molecular weight fraction from the system, i.e. the concentration of polymeric by-products in the discharge from the reaction zone no longer increases significantly. The “high molecular weight fraction” of the polymeric by-products is, for the purposes of the invention, the fraction which is retained by the membrane. The “low molecular weight fraction” of the polymeric by-products is correspondingly the fraction which is capable of passing through the membrane into the permeate (i.e. the separation limit of the membrane used determines what is the low molecular weight fraction and what is the high molecular weight fraction of the polymeric by-products for the purposes of the invention). Further higher molecular weight by-products are formed from the low molecular weight fraction recirculated together with the catalyst to the reaction as a result of further addition of oxirane.

The fractionation by means of a semipermeable membrane in step b) is carried out using a stream which generally comprises at least part of the catalyst comprised in the liquid discharge from the reaction zone and the 1,3-dioxolan-2-one of the general formula I (product) as further components. The catalyst and the product advantageously pass at least partly into the permeate. Preference is given to a process, wherein in addition:
d) the permeate is recirculated at least partly to the reaction zone in step a) and/or the work-up in step b).

The reaction of the oxirane (II) with carbon dioxide in step a) occurs in a reaction zone which can have one or more (e.g. two, three or more than three) reactors. The reactors can be identical or different reactors. These can, for example, each have identical or different mixing characteristics and/or be divided one or more times by internals. Suitable pressure-rated reactors for preparing the 1,3-dioxolan-2-ones of the formula I are known to those skilled in the art. They include the generally customary reactors for gas-liquid reactions, e.g. tube reactors, shell-and-tube reactors, gas recycle reactors, bubble columns, loop apparatuses, stirred vessels (which can also be configured as cascades of stirred vessels), air-lift reactors, etc.

A suitable process for the reaction in a two-part reactor in which the reaction occurs with backmixing to an oxirane conversion of at least 80% in the first part and the reaction occurs under nonbackmixing conditions in the second part, with the carbon dioxide being conveyed in countercurrent to the oxirane through the entire reaction zone, is described in DE 19819586, which is hereby fully incorporated by reference.

The temperature in the reaction in step a) is generally from about 60 to 160° C., preferably from 70 to 150° C., particularly preferably from 90 to 145° C. When the reaction is carried out in more than one reactor, the temperature in each subsequent reactor can be set to a different value than in the preceding reactor. In a specific embodiment, the respective subsequent reactor is operated at a higher temperature than the preceding reactor. In addition, each reactor can have two or more reaction zones which are operated at different temperatures. Thus, for example, a temperature which is different, preferably higher, than that in the first reaction zone can be set in a second reaction zone or a temperature higher than that in a preceding reaction zone can be set in each subsequent reaction zone, e.g. to achieve very complete conversion.

The reaction pressure in step a) is generally from about 2 to 50 bar, particularly preferably from 5 to 40 bar, in particular from 10 to 30 bar. If desired, in the case of a plurality of reactors being used, a pressure which is different (preferably higher) than that in the preceding reactor can be set in each subsequent reactor.

The starting materials carbon dioxide and oxirane can be conveyed in cocurrent or in countercurrent through the reaction zone. An embodiment in which carbon dioxide and oxirane are conveyed in cocurrent through one part of the reaction zone and in countercurrent in another part is also possible. Preference is given to carbon dioxide and oxirane being conveyed in countercurrent through the entire reaction zone.

According to the invention, a liquid discharge is taken from the reaction zone and used for the subsequent work-up. In addition, a gaseous discharge can be taken off at the top of the reactor or, in the case of a reaction zone comprising a plurality of reactors, of one of the reactors. This comprises unreacted carbon dioxide and also possibly further gaseous constituents such as oxirane (II) and/or inerts (noble gases, nitrogen). The gaseous discharge can, if desired, be partly or fully recirculated to the reaction zone. If desired, the gaseous discharge can also be partly or entirely removed from the system in order to avoid accumulation of inert gaseous constituents in the reaction zone.

As catalysts for the process of the invention, it is possible to use catalysts which are known from the literature, e.g. from U.S. Pat. No. 2,773,070, U.S. Pat. No. 2,773,881, Chem. Lett. (1979) p. 1261, Chem. Lett. (1977) P. 517, DE-A 3529263, DE-B 1169459, EP-A 069494 or EP-B 543249, for such reactions. Preference is given to using onium salts or metal salts or mixtures thereof as catalysts.

Suitable onium salts are in principle all compounds of this type, in particular ammonium, phosphonium and sulfonium salts of the general formulae IIIa to IIIc

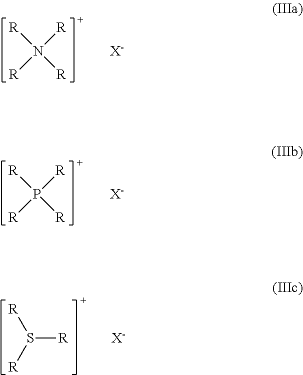

where the substituents R are identical or different hydrocarbon radicals each having from 1 to 20 carbon atoms, with the sum of the carbon atoms in the radicals R being not greater than 24 in each case, and X is an anion equivalent, preferably halide, in particular bromide or iodide.

Preference is given to ammonium salts of the formula IIIa, in particular tetraethyl-ammonium bromide. In addition, preference is given to those compounds IIIa in which three of the radicals R are C₁-C₄-alkyl groups such as methyl or ethyl and the fourth radical R is benzyl or unbranched C₆-C₁₈-alkyl.

Further preferred catalysts are phosphonium salts IIIb which are derived from triphenylphosphine and whose fourth substituent has been introduced into the molecule by quaternization with a C₁-C₆-alkyl bromide.

A suitable sulfonium salt IIIc is, for example, the easily prepared trimethylsulfonium iodide. In general, the ammonium and phosphonium salts are better suited than the sulfonium salts.

In general, the hydrocarbon radicals R in the compounds IIIa to IIIc can be branched or preferably unbranched C₁-C₂₀-alkyl groups, arylalkyl groups such as benzyl groups, the cyclohexyl group and aromatic groups such as the phenyl or the p-tolyl group. Furthermore, alkyl radicals R can also be joined to one another, for instance to form a piperidine ring. Possible anions are halide and also, for example, sulfate and nitrate.

Frequently, and particularly in the case of the onium bromides, it is not necessary to start out from the salts IIIa to IIIc themselves but it is sufficient to use their precursors, viz. base and quaternizing reagent, from which the active quaternization products IIIa to IIIc are formed in situ.

Possible metal salts are salts of alkali metals, alkaline earth metals and transition metals, in particular divalent transition metals, for example sodium, potassium, magnesium, calcium, aluminium, manganese(II), iron(II), nickel(II), copper(II), zinc, cadmium or lead(II) salts. Suitable anions for these salts are sulfate, nitrate, phosphate, carbonate, acetate, formate and especially halides such as chloride, bromide and iodide. Particularly good results are achieved using zinc salts such as zinc sulfate, zinc nitrate, zinc phosphate, zinc carbonate, zinc acetate, zinc formate, zinc chloride, zinc bromide or zinc iodide. It is of course also possible to use mixtures of such metal salts, and the same also applies to the abovementioned onium salts. Mixtures of onium salts with metal salts are also possible and in some cases display surprising advantages.

The amount of the onium salts and/or metal salts used as catalysts is generally not critical. Preference is given to using from about 0.01 to 3% by weight, based on oxirane (II) used.

In a preferred embodiment, alkali metal bromides, alkali metal iodides, tetraalkyl-ammonium bromides, tetraalkylammonium iodides, halides of divalent metals or mixtures thereof are used as catalysts.

In a very particularly preferred embodiment, a mixture of onium salts, in particular ammonium, phosphonium and/or sulfonium salts of the general formulae IIIa to IIIc, and zinc salts, in particular those mentioned explicitly above, is used as catalyst. The effective amounts of the zinc salts here are, depending on the reactivity of the oxirane used, the activity of the onium salt and the other reaction conditions, in the range from 0.1 to 1.0 mol, preferably from 0.3 to 0.7 mol, per mole of onium salt.

Inert solvents suitable for the process of the invention are, for example, dioxane, toluene or acetone. If a solvent is used for the reaction, it is normally used in amounts of from about 10 to 100% by weight, based on the oxirane (II) used. If the process product I is liquid under the reaction conditions, this is advantageously used as solvent, preferably as sole solvent. In such cases, it has been found to be advantageous to dissolve the catalyst in the process product and to meter in this solution, with virtually no further solvents being introduced into the reactor. Here, the concentration of the catalyst in the process product (I) is usually from 0.5 to 20% by weight, in particular from 1 to 15% by weight. The molar ratio of amount of starting material (II) added in the same period of time to process product (I) added with the catalyst is generally from 100:1 to 1:1, in particular from 50:1 to 2:1.

In the process of the invention, the feed streams of oxirane (II) and carbon dioxide are preferably used in a molar ratio of from 1:1 to 1:1.05, in particular from 1:1 to 1:1.02. A possible slight excess of carbon dioxide is advantageous in order to compensate the losses of carbon dioxide on depressurization of the discharge from the reaction zone.

Virtually quantitative conversions of (II), generally at least 99%, in particular at least 99.5%, especially at least 99.9%, are normally achieved by means of the process of the invention.

Suitable radicals R¹ are:
hydrogen,
saturated and unsaturated branched and unbranched aliphatic radicals having up to 40 carbon atoms, in particular up to 18 carbon atoms,
isocyclic or heterocyclic cycloaliphatic groups preferably having from 5 to 7 ring atoms,
isocyclic or heterocyclic aromatic groups and
mixed radicals having groups of the abovementioned type, for example araliphatic radicals such as the benzyl group.

The radicals R¹ which are different from hydrogen can bear one or more substituents such as halogen, nitro groups, free or substituted amino groups, hydroxyl groups, formyl groups or cyano groups or comprise ether, ketone or ester groups. Preference is given to R¹ being hydrogen.

The radicals R² and R³ are generally hydrogen or a methyl group or radicals which are joined to one another to form a five- or six-membered ring, an example of which is cyclohexene oxide as compound II. If II comprises two oxirane rings each having a (CH₂) group, the corresponding bisdioxolanes I are obtained; oxirane rings substituted on both carbon atoms are generally attacked more slowly than those which are substituted on only one of the carbon atoms. Preference is given to using ethylene oxide or propylene oxide, especially ethylene oxide, as oxirane (II).

In a preferred embodiment, ethylene carbonate or propylene carbonate is prepared by means of the process of the invention.

According to the invention, a liquid stream is taken off as discharge from the reaction zone and subjected to a work-up (=step b) of the process of the invention.

The liquid discharge taken off from the reaction zone generally comprises the following constituents:
1,3-dioxolan-2-one of the formula I,
catalyst,
polymeric by-products,
any solvent used for the reaction,
possibly dissolved carbon dioxide,
possibly dissolved oxirane of the formula II.

The work-up in step b) comprises a membrane separation process as essential step. Here, the catalyst used for the reaction and the high boilers formed in the reaction are advantageously separated to such an extent that it is possible to remove a high boiler stream which is low in catalyst or in the ideal case catalyst-free from the system.

The permeate preferably comprises (in the case of a multistage membrane separation based on all stages) at least 70% by weight, particularly preferably at least 80% by weight, in particular at least 90% by weight, of the catalyst present in the stream used for the membrane separation.

The discharge from the reaction zone preferably comprises a proportion of polymeric by-products of not more than 6% by weight, particularly preferably not more than 5% by weight, in particular not more than 4% by weight, based on the total weight of the reaction discharge.

The liquid discharge from the reaction zone is preferably not used directly for the membrane separation in step b) but is instead firstly subjected to removal of part of the components comprised therein. Preference is given to separating off a stream consisting essentially of the compound (I), the catalyst and the polymeric by-products from the discharge from the reaction zone in step b). This stream is then subjected at least partly to the fractionation by means of a semipermeable membrane. In a specific embodiment, this stream is divided into a first substream and a second substream, with the first substream being recirculated to the reaction zone and the second substream being subjected to the fractionation by means of a semipermeable membrane.

Before the membrane separation, carbon dioxide and/or oxirane of the formula II dissolved in the discharge from the reaction zone are at least partly separated off from the discharge from the reaction zone.

Furthermore, preference is given to separating off a stream consisting essentially of the reaction product, i.e. the compound (I), from the discharge from the reaction zone before the membrane separation.

As indicated above, the removal of carbon dioxide and/or oxirane can be carried out via a separate gaseous discharge from the reaction zone. If the process of the invention is configured as a pure liquid discharge process, the discharge from the reaction zone can firstly be subjected to a depressurization step to separate off the carbon dioxide and/or oxirane (II) dissolved therein. This is generally followed by fractionation into a liquid phase consisting essentially of the compound (I), polymeric by-products, the homogeneously dissolved catalyst and possibly small amounts of dissolved carbon dioxide and/or oxirane (II) and a gas phase consisting essentially of carbon dioxide and/or oxirane (II). The gas phase resulting from the depressurization step can be recirculated partly or entirely to the reaction zone. This recirculation can be carried out together with one of the gas streams fed into the reaction zone or separately. The liquid phase obtained in the depressurization step is preferably subjected to a further fractionation by a customary method known to those skilled in the art. The liquid phase is preferably subjected to a distillation to give a stream consisting essentially of the compound (I) and a stream consisting essentially of the compound (I), the catalyst and the polymeric by-products. The latter stream can then be used for the membrane separation.

This stream preferably comprises a proportion of high-boiling by-products of not more than 30% by weight, preferably not more than 25% by weight, particularly preferably not more than 20% by weight, based on the total weight of the stream consisting essentially of the compound (I), the catalyst and the polymeric by-products.

In a specific embodiment, the latter stream is divided into a first substream and a second substream, with the first substream being recirculated to the reaction zone and the second substream being used for the membrane separation.

As an alternative, the discharge from the reaction zone can be subjected directly to a fractionation by distillation into
a stream consisting essentially of unreacted carbon dioxide and/or oxirane (II),
a stream consisting essentially of the compound (I) and
a stream consisting essentially of the compound (I), the catalyst and the polymeric by-products.

The fractionation by distillation of the reaction discharge can be carried out by customary methods known to those skilled in the art. Suitable apparatuses for the fractionation by distillation comprise distillation columns such as tray columns, which can be provided with bubble caps, sieve plates, sieve trays, packings, internals, valves, side offtakes, etc. Dividing wall columns, which may be provided with side offtakes, recirculations, etc., are especially suitable. A combination of two or more than two distillation columns can be used for the distillation. Further suitable apparatuses are evaporators such as thin film evaporators, falling film evaporators, Sambay evaporators, etc, and combinations thereof.

The distillation is preferably carried out at a temperature at the bottom in the range from about 30 to 160° C., particularly preferably from 50 to 150° C., in particular from 70 to 140° C.

The distillation can be carried out under atmospheric pressure, superatmospheric pressure or reduced pressure. The pressure in the distillation is preferably in the range from about 0.0005 bar to 1.5 bar, particularly preferably from 0.001 bar to 1.2 bar, in particular from 0.01 bar to 1.1 bar.

To separate off polymeric by-products, a stream which can be obtained from the discharge from the reaction zone and additionally comprises a compound (I) and the catalyst is brought into contact under pressure with a membrane and a permeate (filtrate) comprising the low molecular weight fraction of the polymeric by-products and the dissolved catalyst is taken off on the rear side of the membrane at a lower pressure than that on the feed side. A solution which is more concentrated in the high molecular weight fraction of the polymeric by-products (high-boiling impurities) and is depleted in catalyst is obtained as retentate.

In a preferred embodiment, the fractionation by means of a membrane in step b) is carried out in two or more than two stages (e.g. in 3, 4, 5 or 6).

In a preferred embodiment, the amount of permeate separated off in the membrane fractionation is at least partly replaced by addition of liquid to the retentate. This replacement can be carried out continuously or discontinuously. A membrane separation (ultrafiltration) in which the retained material is not concentrated but in which the amount of permeate separated off is replaced is also referred to as diafiltration. When the fractionation by means of a membrane in step b) is carried out in two or more than two stages, one stage, a part of the stages or all stages can be configured as a diafiltration. If the product (I) is used as solvent for the reaction, the compound (I) is preferably also used as additionally introduced liquid in the diafiltration.

In a further preferred embodiment, the amount of liquid separated off with the permeate in the membrane separation in step b) is not replaced. An ultrafiltration in which the amount of permeate separated off is not replaced will be referred to as concentration for the purposes of the invention. When the fractionation by means of a membrane in step b) is carried out in two or more than two stages, one of the stages, part of the stages or all stages can be configured as a concentration.

In a preferred embodiment, the membrane fractionation comprises a plurality of stages connected in series. Here, the feed stream is fed to a first membrane fractionation (first stage), and the resulting retentate stream is recirculated to the next stage. The retentate stream taken from the last stage is finally subjected to a work-up to obtain a purge stream comprising the high molecular weight components of the polymeric by-products and a stream enriched in compound (I) and/or solvent.

In a specific embodiment, the fractionation by means of a membrane in step b) comprises firstly at least one concentration step and subsequently at least one diafiltration step.

Furthermore, the fractionation by means of a membrane in step b) is preferably carried out continuously.

Suitable semipermeable membranes have a sufficient permeability for the catalyst dissolved homogeneously in the reaction medium. In addition, they have a sufficient retention capability for the high molecular weight fraction of the polymeric by-products comprised in the reaction medium, i.e. they are capable of retaining relatively high molecular weight compounds which are formed, for example, by oligomerization or polymerization of the oxiranes (II).

At least one membrane having a separation limit in the range from 500 to 20 000 dalton, preferably from 750 to 10 000 dalton, in particular from 1000 to 5000 dalton, is used for the membrane fractionation. The mean average pore size of the membrane is generally from 0.8 to 20 nm, preferably from 0.9 to 10 nm, particularly preferably from 1 to 5 nm.

The semipermeable membranes used according to the invention have at least one separation layer which can consist of one or more materials. These materials are preferably selected from among organic polymers, ceramic materials, metals, carbon and combinations thereof. Suitable materials are stable in the feed medium at the filtration temperature. Preference is given to membranes comprising at least one inorganic material.

Suitable ceramic materials are, for example, α-aluminium oxide, zirconium oxide, titanium dioxide, silicon carbide and mixed ceramic materials.

Suitable organic polymers are, for example, polypropylenes, polytetrafluoroethylenes, polyvinylidene difluorides, polysulfones, polyether sulfones, polyether ketones, polyamides, polyimides, polyacrylonitriles, regenerated cellulose, silicone polymers.

Particular preference is given to using an inorganic membrane made up of a plurality of layers in the process of the invention.

For mechanical reasons, the separation layers are generally applied to a single-layer or multilayer porous substrate composed of the same material as the separation or else a plurality of different materials. Examples of possible material combinations are shown in the following table:

| Separation layer | substrate (coarser than separation layer) |
|---|---|
| Metal | metal |
| Ceramic | metal, ceramic or carbon |
| Polymer | polymer, metal, ceramic or ceramic on metal |
| Carbon | carbon, metal or ceramic |

Ceramic: e.g. α-Al₂O₃, ZrO₂, TiO₂, SiC, mixed ceramic materials
Polymer: e.g. PP, PTFE, PVDF, polysulfone, polyether sulfone, polyether ether ketone, polyamide, polyacrylonitrile, regenerated cellulose

Particular preference is given to separation layers composed of ceramic.

The membranes can in principle be used in flat, tubular, multichannel elements, capillary or wound geometry, for which appropriate pressure housings which allow separation between retentate and permeate are available.

The optimal transmembrane pressures between retentate and permeate are dependent on the diameter of the membrane pores, the hydrodynamic conditions, which influence the structure of the covering layer, and the mechanical stability of the membrane at the filtration temperature. They are generally in the range from 0.2 to 30 bar, particularly preferably in the range from 0.5 to 20 bar. Higher transmembrane pressures generally lead to higher permeate fluxes. If a plurality of modules are connected in series, the transmembrane pressure for each module can be reduced by increasing the permeate pressure and thus matched to the membrane. The operating temperature is dependent on the membrane stability and the thermal stability of the feed. A suitable temperature range for the membrane separation in step b) is from 20 to 90° C., preferably from 40 to 80° C. The melting points of the products can limit the temperature range. Higher temperatures generally lead to higher permeate fluxes. The achievable permeate fluxes are greatly dependent on the type of membrane and membrane geometry used, on the process conditions, on the feed composition (essentially the polymer concentration). The fluxes are typically in the range from 0.5 to 100 kg/m²/h, preferably from 1 to 50 kg/m²/h.

The following membranes, for example, can be used:

| | | Separation |
|---|---|---|
| | | limit (kD) pore |
| Manufacturer | Membrane | diameter (nm) |
| Inopor GmbH | γ-Al₂O₃ on ceramic/1, 2 | 5 nm; 7.5 kD |
| | TiO₂ on ceramic/1, 2 | 5 nm; 8.5 kD |
| | TiO₂ on ceramic/1, 2 | 0.9 nm; 0.5 kD |
| | TiO₂ on ceramic/1, 2 | 1 nm; 0.8 kD |
| | ZrO₂ on ceramic/1, 2 | 3 nm; 2 kD |
| Atech inno- | UF/TiO₂ on α-Al₂O₃/1, 2 | 5, 10 and 20 kD |
| vations GmbH | | |
| Rhodia/Orelis | UF/ZrO₂ or TiO₂ on ceramic/1, 2 | 15 kD |
| Pall-Schumacher | UF/TiO₂ or ZrO₂ on ceramic/1, 2 | 5 and 10 nm |
| Creavis | UF/ZrO₂ on α-Al₂O₃ and metal/3 | 25 nm |

1: Tubular membrane;
2: multichannel element;
3: flat membrane for wound, bag, plate stack or special modules having an agitated membrane or stirrers between the membranes

The membrane separation in step b) can be carried out discontinuously even in the case of otherwise continuous operation of the reaction, for example by multiple passage through the membrane modules. The membrane separation in step b) is preferably carried out continuously, for example by means of a single pass through one or more membrane separation stages connected in series.

To avoid an appreciable buildup of a covering layer of retained high molecular weight fraction of the polymeric by-products on the membrane surface, which can lead to a decrease in the permeate flux, pumped circulation, mechanical agitation of the membrane or the use of stirrers between the membranes has been found to be useful. These measures serve to generate a relative velocity between membrane and reaction discharge to be separated in the range from 0.1 to 10 m/s.

The high-boiling impurities can be separated off from the retentate by methods known per se. The retentate is preferably subjected to a distillation to give a purge stream enriched in high-boiling compounds and a stream enriched in compound (I). The distillation can be carried out using apparatuses known per se, e.g. by use of at least one short path evaporator.

The invention is illustrated below with the aid of figures which represent preferred embodiments of the process of the invention, without the invention being restricted thereto.

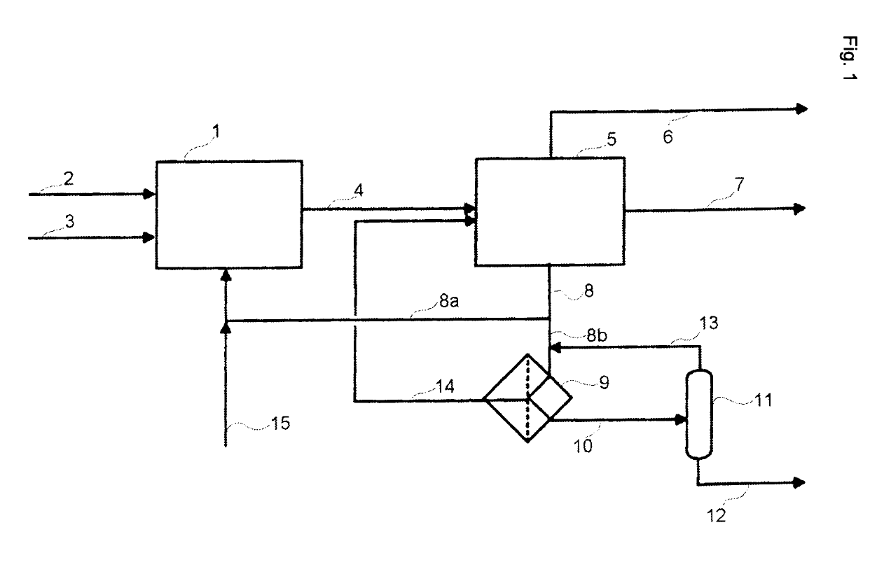

FIG. 1 shows a schematic representation of a plant suitable for carrying out the process according to the invention.

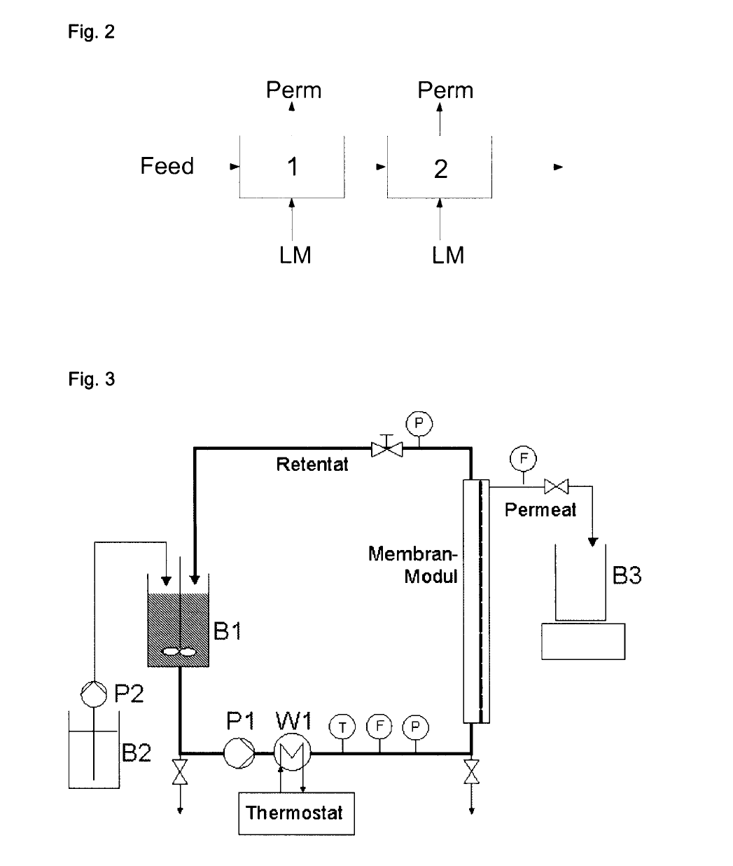

FIG. 2 shows a schematic representation of a continuously operated two-stage membrane cascade.

FIG. 3 shows a schematic representation of the apparatus used in the examples.

FIG. 1 represents a scheme of a plant suitable for carrying out the process according to the invention, with details which are not relevant for explaining the invention having been omitted for reasons of clarity. The plant comprises a reaction zone (1) comprising at least one reactor. An oxirane (e.g. ethylene oxide) is introduced into the reaction zone (1) via line (2) and CO₂ is introduced into the reaction zone (1) via line (3). A discharge (4) is taken off from the reactor (1) and brought to the work-up stage (5) via the line coming off the reaction zone (1). In a specific embodiment, the discharge (4) is firstly introduced into a depressurization vessel (not shown) in which phase separation into a gas phase comprising carbon dioxide and a liquid phase occurs. The liquid phase is subsequently passed to a further work-up in the work-up stage (5). The gas phase obtained in the depressurization can additionally comprise proportions of unreacted oxirane. In the work-up stage (5), a fractional distillation is carried out to give a gas phase (6) comprising the low-boiling components of the reaction discharge (i.e. essentially carbon dioxide and/or oxirane), a stream (7) consisting essentially of the 1,3-dioxolan-2-one (e.g. ethylene carbonate) and a bottom product (8) consisting essentially of the 1,3-dioxolan-2-one, the catalyst and the polymeric by-products of the reaction. The stream (8) comprising the catalyst and the polymeric by-products is divided into a first substream (8a) which is recirculated to the reaction zone (1) and a second substream (8b) which is fed to the membrane separation (9). The membrane separation (9) can have one or more stages. The membrane separation (9) produces a retentate stream (10) which comprises the high-boiling components of the reactor discharge retained by the semipermeable membrane and also oxirane and possibly small proportions of catalyst which has not been separated off. This retentate stream (10) is fed to a work-up stage (11) which, in a specific embodiment, is configured as a short path evaporator. The high boiler stream (12) obtained in the work-up stage (11) is removed from the process. The 1,3-dioxolan-2-one-enriched stream (13) which is likewise obtained is recirculated to the membrane separation (9). The permeate (14) obtained in the membrane separation (9), which consists essentially of the 1,3-dioxolan-2-one, the catalyst and the proportions of high boilers which have not been retained in the membrane separation (9), is recirculated, in a first embodiment, to the work-up stage (5). In a second embodiment (not shown), the permeate stream (14) is recirculated to the reaction zone (1). Fresh catalyst can, if necessary, be fed into the reaction zone (1) via the feed stream (15).

FIG. 2 schematically shows a preferred embodiment of the fractionation of a stream comprising catalyst and high boilers by means of a continuously operated two-stage membrane cascade, with the two stages being able to be configured as a concentration step (solvent stream=0) or diafiltration step (solvent stream=permeate stream) or as a mixed form of concentration and diafiltration steps (0<solvent stream<permeate stream).

Definitions for continuously operated membrane stages based on one stage:

Sol=solvent
Perm=permeate
Diafiltration: =Sol stream=Perm stream
Concentration: =Sol stream=0
Mixed form comprising=0<Sol stream<Perm stream
diafiltration and concentration:

The definitions of concentration and diafiltration differ for the continuous mode of operation depicted and the batch mode of operation which is likewise possible:

continuous operation:
diafiltration: solvent stream=permeate stream
mixed form: 0<solvent stream<permeate stream
concentration: solvent stream=0
batch operation:
diafiltration: solvent stream=permeate stream
concentration: solvent stream=0

FIG. 3 schematically shows the apparatus which is used in the examples and is operated batchwise.
B1=circulation vessel
B2=reservoir for diafiltration medium
B3=permeate collection vessel (for permeate flow measurement on balance)
P1=centrifugal pump
P2=metering pump
W1=heat exchanger
P=pressure measurement
T=temperature measurement
F=flow measurement

The invention will be illustrated by the following, nonlimiting examples.

The experiments on separating off the high molecular weight fraction of the polymeric by-products from the catalyst and the low molecular weight fraction of the polymeric by-products were carried out using a ceramic multilayer membrane from Inopor GmbH, which had a zirconium dioxide separation layer having an average pore diameter of 3 nm and a molecular separation limit of 2 kD. A 19/3.5 multichannel element (19 holes having an internal diameter of 3.5 mm, on the inside of which the membrane has been applied, are present in an element) having a length of 50 cm and an area of 0.098 m² was used. Catalyst-comprising reactor discharges from the synthesis of ethylene carbonate (catalyst: bromide salt mixture) which had been freed of low boilers by distillation and from which ethylene carbonate had been partly separated off by distillation were used.

This ethylene carbonate-, catalyst- and polymer-comprising feed was worked up batchwise in all experiments. For this purpose, the material used was brought from a circulation vessel to a pressure of 15 bar by means of a pump and passed at a temperature of 70° C. and a velocity of 2 m/s through the membrane tubes, then depressurized to atmospheric pressure again and fed back into the circulation vessel. Permeate separated off at atmospheric pressure was collected in a vessel on a balance in order to determine the permeate flux and was continuously replaced by an equal amount of diafiltration medium (ethylene carbonate in all experiments). Diafiltration was generally carried out at a solvent exchange coefficient MA of about 3, i.e. at an amount of feed of × kg, 3× kg of permeate were taken off and 3× kg of ethylene carbonate were added to the retentate so that the amount of retentate remained constant. At the end of the experiments, the feeds and the retentates were analyzed. The common anion of the catalyst salt mixture (bromide) was used for the catalyst analysis.

| | Average | | Feed | | | Retentate | | Yield of | |
|---|---|---|---|---|---|---|---|---|---|
| | permeate | | M_{W} | Concentration | | Concentration | | retentate | |
| | flow | | HB | Br⁻ | | Br⁻ | | Br⁻ | HB |
| Experiment | (kg/m²/h) | MA | (kg/mol) | (%) | HB (%) | (%) | HB (%) | (%) | (%) |
| 1 | 8.6 | 3.01 | 10 | 1.215 | 23.2 | 0.079 | 14.3 | 6.5 | 62 |
| 2 | 13.4 | 2.93 | 7 | 1.438 | 14.1 | 0.184 | 7.0 | 12.8 | 50 |

M_{w}: weight average molecular weight (GPC)
HB: high boilers
Br⁻: bromide (potentiometric titration)

The experiments shown that the catalyst and the low molecular weight fraction of the polymeric by-products can be removed from the catalyst- and polymer-comprising feed and that the higher molecular weight fraction of the polymeric by-products is retained by the membrane. It is thus possible to provide a stream which is low in catalyst and comprises polymer and ethylene carbonate for removal of the relatively high molecular weight fraction of the polymeric by-products.

1. A process for the continuous preparation of a 1,3-dioxolan-2-one of the general formula I 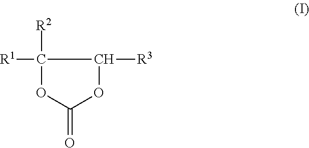
where
R¹ is hydrogen or an organic radical having from 1 to 40 carbon atoms, R² and R³ are each, independently of one another, hydrogen or C₁-C₄-alkyl, where R² and R³ may also be joined to one another to form a five- or six-membered ring, wherein comprising:
a) reacting an oxirane of the general formula II 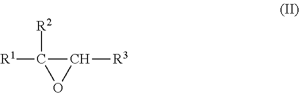
where R₁, R² and R³ are as defined above, with carbon dioxide in the liquid phase in the presence of a catalyst dissolved homogeneously in the liquid phase in a reaction zone,
b) taking a liquid discharge comprising polymeric by-products of the reaction from the reaction zone, and subjecting the liquid discharge to a work-up comprising fractionation by means of a semipermeable membrane to give a permeate and a retentate, with a high molecular weight fraction of the polymeric by-products being retained by the membrane, when the fractionation comprises passing a stream comprising catalyst in addition to the polymeric by-products through a semipermeable membrane, with the catalyst passing at least partly into the permeate, and
c) obtaining a purge stream comprising the high molecular weight fraction of the polymeric by-products.

2. The process according to claim 1, wherein a low molecular weight fraction of the by-products passes into the permeate.

3. The process according to claim 1, wherein, in addition:
d) the permeate is recirculated at least partly to the reaction zone in a) and/or the work-up in b).

4. The process according to claim 1, wherein a stream consisting essentially of the compound (I), the catalyst and the polymeric by-products is separated off from the discharge from the reaction zone in b) and is subjected at least partly to the fractionation by means of a semipermeable membrane.

5. The process according to claim 4, wherein the stream consisting essentially of the compound (I), the catalyst and the polymeric by-products which has been separated off from the discharge from the reaction zone is at least partly recirculated to the reaction zone.

6. The process according to either claim 4, wherein the stream consisting essentially of the compound (I), the catalyst and the polymeric by-products is obtained by separating off a gaseous stream consisting essentially of unreacted carbon dioxide and/or the oxirane of the formula (II) and a stream consisting essentially of the compound (I) from the discharge from the reaction zone.

7. The process according to claim 6, wherein the discharge from the reaction zone is firstly subjected to depressurization in which fractionation into a liquid phase consisting essentially of the compound (I), polymeric by-products, the homogeneously dissolved catalyst and optionally small amounts of dissolved carbon dioxide and/or oxirane (II) and a gas phase consisting essentially of carbon dioxide and/or oxirane (II) occurs and the liquid phase is subjected to a distillation to give a stream consisting essentially of the compound (I) and a stream consisting essentially of the compound (I), the catalyst and the polymeric by-products.

8. The process according to claim 6, wherein the discharge from the reaction zone is subjected to a fractional distillation into
a first stream consisting essentially of unreacted carbon dioxide and/or oxirane (II),
a second stream consisting essentially of the compound (I) and
a third stream consisting essentially of the compound (I), the catalyst and the polymeric by-products.

9. The process according to claim 1, wherein the fractionation by means of a membrane in b) is carried out in two or more stages.

10. The process according to claim 9, wherein the fractionation further comprises diafiltration, wherein the amount of liquid separated off with the permeate is at least partly replaced on the retentate side by additionally introduced liquid.

11. The process according to claim 10, wherein the additionally introduced liquid comprises compound (I).

12. The process according to claim 9, wherein the fractionation in b) further comprises concentrating, wherein the liquid separated off with the permeate is not replaced by additionally introduced liquid.

13. The process according to claim 9, wherein the fractionation in b) comprises a mixed form comprising diafiltrating and concentrating in which the amount of liquid separated off with the permeate is partly replaced by the additionally introduced liquid.

14. The process according to claim 9, wherein the stages of the membrane fractionation are connected in series.

15. The process according to claim 9, wherein the fractionation by means of a membrane comprises firstly concentrating and subsequently diafiltrating.

16. The process according to claim 1, wherein the fractionation by means of a membrane in b) is carried out continuously.

17. The process according to claim 1, wherein the membrane of the membrane fractionation is at least one membrane having a separation limit in the range from 500 to 20 000 dalton.

18. The process according to claim 1, wherein the membrane of the membrane fractionation is at least one membrane having a pore diameter in the range from 0.8 to 20 nm.

19. The process according to claim 1, wherein the membrane of the membrane fractionation is a polymer membrane or an inorganic membrane.

20. The process according to claim 1, wherein the membrane of the membrane fractionation is at least one inorganic membrane comprising a plurality of layers.

21. The process according to claim 1 for preparing ethylene carbonate or propylene carbonate.
